(19) United States
(12) Patent Application Publication
(10) Pub. No.: US 2014/0081360 A1
(43) Pub. Date: Mar. 20, 2014

(54) CAPSULE PHOTOTHERAPY

(71) Applicants: Sharon BEN-YEHUDA, Rechovot (IL); Ram BEN-YEHUDA, Rechovot (IL)

(72) Inventors: Sharon BEN-YEHUDA, Rechovot (IL); Ram BEN-YEHUDA, Rechovot (IL)

(73) Assignee: PhotoPill Medical Ltd., Rechovot (IL)

(21) Appl. No.: 13/623,177

(22) Filed: Sep. 20, 2012

(51) Int. Cl. A61N 5/06

(52) U.S. Cl. 607/92

(57) ABSTRACT

A method for providing phototherapy to a GI tract including using a capsule imager to scan at least a portion of a GI tract, estimating a location of a diseased portion of the GI tract, and based, at least in part, on the estimated location, using a phototherapy capsule to provide phototherapeutic light to the diseased portion.

A swallowable capsule for providing phototherapy to a GI tract including a power supply, a camera unit, one or more light sources emitting in a range including visible and Near Infra Red (NIR), and a control unit for calculating an effective dose to the GI tract by controlling the one or more light sources, a communication unit for transmitting images from the camera unit to an out-of-body receiver and for receiving control commands from an out-of-body transmitter.

Related apparatus and methods are also described.

### RELATED APPLICATIONS

This application claims the benefit of priority under 35 USC §119(e) of U.S. Provisional Patent Application No. 61/537,121 filed on Sep. 21, 2011, the contents of which are incorporated herein by reference in their entirety.

This application is related to PCT Patent Application No. PCT/IL2011/000257 having International filing date of Mar. 17, 2011, which claims the benefit of priority under 35 USC §119(e) of U.S. Provisional Patent Application No. 61/314,762 filed on Mar. 17, 2010. The contents of the above applications are all incorporated herein by reference.

### FIELD AND BACKGROUND OF THE INVENTION

The present invention, in some embodiments thereof, relates to capsule phototherapy combined with capsule endoscopy and, more particularly, but not exclusively, to using capsule imaging for diagnostics and capsule phototherapy for treatment, and in particular, for treating inflammatory bowel disease (IBD).

Capsule endoscopy is used to examine parts of the gastrointestinal (GI) tract, especially parts that cannot be seen with other types of endoscopy. Capsule endoscopy is useful when disease is detected in the small intestine, and can sometimes diagnose sources of occult bleeding (blood visible macroscopically only) or causes of abdominal pain such as Crohn's disease, or peptic ulcers. Capsule endoscopy can be used to diagnose problems in the small intestine, but unlike EGD or colonoscopy, typically does not treat pathology that may be discovered. Capsule endoscopy may be performed by recording and subsequent analysis, and/or by real-time viewing.

Light therapy, conventionally referred to as “phototherapy”, consists of exposure to daylight or to specific wavelengths of light using lasers, light-emitting diodes, fluorescent lamps, dichroic lamps or very bright, full-spectrum light, usually controlled with various devices. The light is administered for a prescribed amount of time and, in some cases, at a specific time of day.

Phototherapy is generally applied to relatively easily accessible tissue regions, such as external regions of the skin and the mucosa lining the mouth or nose, and is used to treat acne, psoriases, eczema, vitiligo (in which damage to skin pigment cells results in white skin patches) and skin-based lymphoma, gingivitis, gum inflammations, oral ulcers, and allergic rhinitis.

A similar-sounding but different field from phototherapy is Photodynamic therapy (PDT). Most modern PDT applications involve a production of a chemical reaction in tissue, caused by light, where light interacts with a chemical injected to the body. The wavelength of the light source needs to be appropriate for exciting chemical reaction. In understanding the mechanism of PDT it is important to distinguish it from other light-based and laser therapies such as laser wound healing and rejuvenation which do not require interaction with an externally administered chemical.

International Patent Application Publication WO 2008/012701 is entitled “Capsule camera with variable illumination of the surrounding tissue”, and discloses an ingestible capsule and method for in vivo imaging and/or treatment of one or more diseased areas of interest within the gastrointestinal tract of an animal or human being. The capsule comprises an image sensor; a lens system for focusing images onto the image sensor; at least one light source for illumination of the tissue area of interest, the at least one light source optionally being capable of providing optical therapeutic treatment to the diseased areas; a variable lens system located in front of the at least one light source, wherein the variable lens system comprises beam steering means and focusing means for directing and focusing the light beams from the at least one light source onto the diseased tissue areas, —a control unit in communication with the image sensor, the at least one light source, and variable lens system, a power source for powering the image sensor, the at least one light source and the control unit; and a non-digestible, transparent outer protective shell configured to pass through the gastrointestinal tract, housing within the image sensor, the lens system, the at least one light source, the variable lens system, the control unit and the power source.

An article entitled “Autonomous Device for Photostimulation of the Gastrointestinal Tract Immunity” by Sergey A. Naumov Vladimir N. Dyrin, Sergey M. Vovk, Evgeny Y. Petrov, Vladimir V. Udut and Elena V. Borodulina, published in Proc. SPIE 3907, 433 (2000); doi:10.1117/12.386284, describes a very small optoelectronic device emitting light in the red and green band has been developed as a small capsule consisting of two semispheres connected with light-transmitted coupling. The device—a phototablet permits to irradiate all parts of the gastrointestinal tract (GIT) including the immunocompetent formations of the small intestine—Peyer's patches responsible for production of secretory immunoglobulins A (IgA). The main mechanisms of realizing endogenic phototherapy using a phototablet begin functioning when irradiating both the walls of the GIT organs and its contents. The results of clinical trials of the phototablet testify to a favorable effect of endogenic therapy on the human organism in asthenic syndrome, some types of deficiency in the immunity function, in dysbioses, the syndrome of large intestine irritation, duodenostasis, etc. After endogenic phototherapy the patients had an increased level of lysozyme, leukocytes, a number of lactobacteria. There were no side effects when using a phototablet. Indications and contraindications for endogenic phototherapy were represented. Thus, the method of endogenic phototherapy allows us to have an effective and direct influence on the immunocompetent cells of GIT organs without medicamental agents and antigens that makes it possible to use the phototablet in medicine on a large scale.

US published patent application 2009/0177033 of Hendriks at al., describes an application which relates to an ingestible capsule and method for in vivo imaging and/or treatment of one or more diseased areas of interest within the gastrointestinal tract of an animal or human being. The capsule comprises an image sensor; a lens system for focusing images onto the image sensor; at least one light source for illumination of the tissue area of interest, the at least one light source optionally being capable of providing optical therapeutic treatment to the diseased areas; a variable lens system located in front of the at least one light source, wherein the variable lens system comprises beam steering means and focusing means for directing and focusing the light beams from the at least one light source onto the diseased tissue areas, a control unit in communication with the image sensor, the at least one light source, and variable lens system, a power source for powering the image sensor, the at least one light source and the control unit; and a non-digestible, transparent outer protective shell configured to pass through the gastrointestinal tract, housing within the image sensor, the lens system, the at least one light source, the variable lens system, the control unit and the power source.

Additional background art includes:

PCT published patent application WO 2009/102445 of Bandy et al;

U.S. published patent application 2008/0106596 of Iddan et al;

U.S. published patent application 2004/0249245 of Irion;

U.S. published patent application 2004/0106849 of Cho et al;

U.S. published patent application 2003/0214579 of Iddan; and

U.S. Pat. No. 5,464,436 to Smith.

### SUMMARY OF THE INVENTION

The present invention, in some embodiments thereof, combines capsule endoscopy for diagnosing and/or mapping which part of a GI tract should receive capsule phototherapy, with capsule phototherapy.

The term “capsule imager” in all its grammatical forms is used throughout the present specification and claims to stand for a capsule which includes a camera.

The term “phototherapy capsule” in all its grammatical forms is used throughout the present specification and claims to stand for a capsule which includes one or more sources of light for providing phototherapy.

According to an aspect of some embodiments of the present invention there is provided a method for providing phototherapy to a GI tract including using a capsule imager to scan at least a portion of a GI tract, estimating a location of a diseased portion of the GI tract, and based, at least in part, on the estimated location, using a phototherapy capsule to provide phototherapeutic light to the diseased portion.

According to some embodiments of the invention, a medical practitioner programs the phototherapy capsule to provide phototherapeutic light to the diseased portion based, at least in part, on the estimating a location providing a time required for the phototherapy capsule to reach a diseased portion of the GI tract.

According to some embodiments of the invention, the estimating a location is performed automatically, and further including automatically programming the phototherapy capsule to provide phototherapeutic light to the diseased portion based, at least in part, on the estimating a location.

According to some embodiments of the invention, the estimating a location includes automatic lesion detection in images captured by the capsule imager. According to some embodiments of the invention, the estimating a location includes analyzing tissue color in images captured by the capsule imager. According to some embodiments of the invention, the estimating a location includes detecting local bleeding in images captured by the capsule imager.

According to some embodiments of the invention, a medical practitioner selects a phototherapy capsule which is pre-programmed for approximately a time required for the phototherapy capsule to reach a diseased portion of the GI tract.

According to some embodiments of the invention, the using a capsule imager and the estimating a location of a diseased portion of the GI tract are performed in a medical clinic, and the using a phototherapy capsule to provide phototherapeutic light to the diseased portion is performed by a patient in a non-clinic setting.

According to some embodiments of the invention, the phototherapy capsule determines when to turn on and off one or more phototherapeutic light sources based, at least in part, on the estimating a location providing a time required for the phototherapy capsule to reach a diseased portion of the GI tract.

According to some embodiments of the invention, the capsule imager further records data about the environment in the GI tract, the phototherapy capsule measures data about the environment in the GI tract, the phototherapy capsule determines when to turn on and off one or more phototherapeutic light sources based, at least in part, on the data about the environment which the capsule imager recorded and the phototherapy capsule measures.

According to some embodiments of the invention, measuring data about the environment in the GI tract includes pH measurement. According to some embodiments of the invention, measuring data about the environment in the GI tract includes measuring ultrasound from an ultrasound beacon. According to some embodiments of the invention, measuring data about the environment in the GI tract includes measuring sound from a sonic beacon.

According to some embodiments of the invention, the data recorded includes location data, and in which the phototherapy capsule determines when to turn on and off one or more phototherapeutic light sources based, at least in part, on the phototherapy capsule reaching a location of a diseased portion of the GI tract.

According to some embodiments of the invention, the phototherapy capsule determines when to turn on and off one or more phototherapeutic light sources in order to provide a required treatment energy to the diseased portion of the GI tract.

According to some embodiments of the invention, the required treatment energy is computed, at least in part, based on speed of movement of the phototherapy capsule in the GI tract.

According to some embodiments of the invention, the phototherapy capsule determines when to turn on and off one or more phototherapeutic light sources based, at least in part, on the phototherapy capsule measuring a speed of movement of the phototherapy capsule in the GI tract.

According to some embodiments of the invention, the phototherapy capsule adjusts an intensity of a light source based, at least in part, on the phototherapy capsule measuring a speed of movement of the phototherapy capsule in the GI tract.

According to some embodiments of the invention, the phototherapy capsule uses the capsule imager to measure an intensity of the phototherapeutic light provided to the diseased portion.

According to some embodiments of the invention, the phototherapy capsule calculates a dose provided to the diseased portion of the GI tract based, at least in part, on the measured intensity and on speed of movement of the phototherapy capsule in the GI tract.

According to some embodiments of the invention, the providing phototherapeutic light to the diseased portion comprises in addition providing light for PhotoDynamic Therapy (PDT). According to some embodiments of the invention, the providing phototherapeutic light to the diseased portion comprises instead providing light for PhotoDynamic Therapy (PDT).

According to an aspect of some embodiments of the present invention there is provided a swallowable capsule for providing phototherapy to a GI tract including a power supply, a camera unit, one or more light sources emitting in a range including visible and Near Infra Red (NIR), and a control unit for calculating an effective dose to the GI tract by controlling the one or more light sources, a communication unit for transmitting images from the camera unit to an out-of-body receiver and for receiving control commands from an out-of-body transmitter.

According to some embodiments of the invention, further including a communication unit for transmitting images from the camera unit to an out-of-body receiver and for receiving control commands from an out-of-body transmitter.

According to some embodiments of the invention, further including a magnet for manipulating the capsule within the GI tract.

According to some embodiments of the invention, the one or more light sources include a plurality of light sources, each providing light to a substantially limited angle around a longitudinal axis of the capsule, and the control unit is configured to turn at least one of the light sources off, and at the same time at least one of the light sources is on, based, at least in part, on automatically identifying whether the limited angle which the light source illuminates includes a diseased portion of the GI tract.

According to some embodiments of the invention, the one or more light sources include a plurality of light sources, each providing light to a substantially limited angle around a longitudinal axis of the capsule, and the control unit is configured to at turn least one of the light sources off, and at the same time at least one of the light sources on, based, at least in part, on receiving instructions communicated from an external transmitter.

According to an aspect of some embodiments of the present invention there is provided a method for providing phototherapy to a GI tract including using a capsule which includes a capsule imager and a phototherapy capsule, to provide images from the GI tract, identifying a diseased portion of the GI tract, using one or more light sources to provide phototherapeutic light at a location corresponding to the diseased portion of the GI tract, based, at least in part, on the images, in which the one or more light sources are controlled in order to provide an effective dose to the diseased portion of the GI tract.

According to some embodiments of the invention, the capsule additionally includes a magnet, and further including a medical practitioner manipulating the capsule to the diseased portion using an external magnet.

According to some embodiments of the invention, the identifying is performed automatically and further including automatically programming the phototherapy capsule to provide phototherapeutic light to the diseased portion based, at least in part, on the identifying.

According to some embodiments of the invention, the identifying includes automatic lesion detection in images captured by the capsule imager. According to some embodiments of the invention, the identifying includes analyzing tissue color in images captured by the capsule imager. According to some embodiments of the invention, the identifying includes detecting local bleeding in images captured by the capsule imager.

According to some embodiments of the invention, the one or more light sources include a plurality of light sources, each providing light to a substantially limited angle around a longitudinal axis of the capsule, and causing at least one of the light sources to be off, while causing at least one of the light sources to be off, based, at least in part, on identifying whether the limited angle which the light source illuminates includes a diseased portion of the GI tract.

According to some embodiments of the invention, the one or more light sources are turned on or off based, at least in part, on speed of movement of the capsule in the GI tract. According to some embodiments of the invention, an intensity of a light source is adjusted, based, at least in part, on speed of movement of the phototherapy capsule in the GI tract.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk, and/or flash memory, and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a simplified flow chart illustration of a method of providing phototherapy to a GI tract according to an example embodiment of the present invention;

FIG. 2A is a simplified horizontal bar graph corresponding to a length of a GI tract, a corresponding horizontal bar graph of capsule imager time taken to journey through the GI tract, and a corresponding horizontal bar graph of phototherapy capsule time taken to journey through the GI tract, according to an example embodiment of the invention;

FIG. 2B is a simplified illustration of a directional beacon providing signals to a patient's body, according to an example embodiment of the present invention;

FIG. 2C is a simplified horizontal bar graph corresponding to a length of a GI tract, a corresponding horizontal bar graph of capsule imager time taken to journey through the GI tract, a corresponding graph of frequency “φ” of a directional beacon as measured by a capsule journeying through the GI tract, a corresponding graph of intensity “I” of the directional beacon as measured by a capsule journeying through the GI tract, and a corresponding horizontal bar graph of phototherapy capsule time taken to journey through the GI tract;

FIG. 2D is a simplified horizontal bar graph corresponding to a length of a GI tract, a corresponding horizontal bar graph of capsule imager time taken to journey through the GI tract, a corresponding graph of pH as measured by a capsule journeying through the GI tract, and a corresponding horizontal bar graph of phototherapy capsule time taken to journey through the GI tract, according to an example embodiment of the invention;

FIG. 3A is a simplified block diagram illustration of a swallowable combination capsule for providing capsule endoscopy and phototherapy to a GI tract, constructed according to an example embodiment of the invention;

FIG. 3B is another simplified block diagram illustration of a swallowable combination capsule 350 for providing capsule endoscopy and phototherapy to a GI tract, constructed according to an example embodiment of the invention; and

FIG. 4 is a simplified flow chart illustration of a method of providing phototherapy to a GI tract according to an example embodiment of the present invention.

### DESCRIPTION OF SPECIFIC EMBODIMENTS OF THE INVENTION

The present invention, in some embodiments thereof, relates to capsule phototherapy combined with capsule endoscopy and, more particularly, but not exclusively, to using capsule endoscopy for diagnostics and data collecting and capsule phototherapy for treatment, and in particular, for treating inflammatory bowel disease (IBD). In some embodiments of the invention, parameters derived from a capsule endoscopic sensor are used to control phototherapy.

The present invention, in some embodiments thereof, relates to using some capabilities of a capsule imager, which are not necessarily used for diagnostics, to set some phototherapy treatment parameters, and in particular, for treating inflammatory bowel disease (IBD).

Overview

Some advantages of combining capsule endoscopy for diagnosing and/or mapping and/or data collecting with capsule phototherapy, without excluding other possible advantages, include: providing efficient phototherapy treatment, providing phototherapy treatment only where needed, saving power during phototherapy—so as to enable treatment of more of a diseased area—possibly all of a diseased area, enabling treatment with one phototherapy capsule where—if a diseased area for treatment was not defined—more than one phototherapy capsule would be needed.

Using a Separate Capsule Imager and a Separate Phototherapy Capsule

In some embodiments of the invention a separate capsule imager is used, followed by a separate phototherapy capsule.

The separate capsule imager is swallowed to collect data about the GI tract. The data may be images, and may be other data which is useful for a phototherapy capsule. The data is optionally stored for eventual analysis by a medical practitioner, and/or for automatic generation of a phototherapy treatment plan. The data includes, by way of a non-limiting list:

images of the GI tract;

spectral data of walls of the GI tract, such as tissue color, and/or local bleeding, and/or multi-spectral readings of walls of the GI tract;

environmental parameters, such as pH, temperature;

dimensional parameters, such as location, and/or speed, based on an external beacon signal, and/or based on an accelerometer, and/or based on an optical motion sensor, and/or based on sensors, such as sensors on a vest worn by a patient, triangulating a source of a signal from the capsule imager; and

elapsed time.

When a capsule is used for planning treatment for a person who is already slated for phototherapy, there is a possibility of transmitting fewer images per second, and/or less detailed images, to an external monitor. A capsule imager advances rather slowly in most sections of the GI tract. When the transmitting is used for deciding when and/or where to provide phototherapy, the capsule may transmit at a lower rate, just enough to scan the GI tract, without producing multiple images of the same area. The lower transmission rate and/or image resolution may allow using less expensive, or previous-technology-generation, capsule imagers. The lower transmission rate and/or image resolution may provide a power saving, which may be translated into a less expensive battery.

In some embodiments of the invention, the capsule imager is not used for an exhaustive search, for example for polyps, but rather for a less exhaustive search for large lesions or for finding a statistical fraction of existing lesions. In such embodiments, a lower transmission rate and/or image resolution capsule imager may be used. Optionally, the capsule imager may be induced to move more quickly, optionally by ingesting more fluids, optionally saving time to pass along the GI tract.

In some embodiments of the invention the separate phototherapy capsule or capsules are optionally programmed to provide phototherapy at diseased portions of the GI tract, as detected by capsule imager, and as located by using the data collected by the capsule imager.

In some embodiments of the invention the separate phototherapy capsule or capsules are optionally automatically programmed.

In some embodiments of the invention the separate phototherapy capsule or capsules are optionally selected from pre-programmed phototherapy capsules having pre-programmed treatment parameters suitable for providing phototherapy at diseased portions of the GI tract.

In many cases, providing phototherapy to a large area of a GI tract can take more power than available in a phototherapy capsule battery. More information on calculating how much power is needed to treat a how much area of the GI tract is detailed further below, in sections named “total treatment energy” and “area of treatment”.

When battery power is not enough, or barely enough, for providing power for phototherapy, it is also not enough for providing power for capsule endoscopy, which requires powering a camera and transmitting images outside the patient's body.

At least in such cases, an embodiment of the present invention includes using a capsule imager to scan a GI tract, recording diseased locations in the GI tract, then, based on data describing the diseased locations, using capsule phototherapy to treat the diseased locations. In some embodiments of the invention a medical practitioner uses data from the capsule imager to plan capsule phototherapy by making decisions about the treatment, some examples of which are:
How many phototherapy capsules will be used to cover the diseased locations.
What period of time between treatments.
Programming the phototherapy capsule(s).

It is noted that while most of the descriptions of treatment are described as treatment using phototherapeutic illumination, the methods and apparatus described herein lend themselves well to providing illumination for photodynamic treatment (PDT).

In some embodiments of the invention the separate phototherapy capsule or capsules are optionally programmed to provide illumination for PDT at diseased portions of the GI tract, as detected by capsule imager, and as located by using the data collected by the capsule imager.

Using a Capsule Imager within a Phototherapy Capsule

In some embodiments of the invention, capsule imager functions are packaged together with capsule phototherapy functions in one capsule.

The combined, multi-function capsule is swallowed and passes through the GI tract. The combined capsule collects data, and transmits the data to an external data collector, and/or performs analysis and therapy automatically using resources in the capsule. The data may be images, and may be other data which is useful for detecting a GI tract disease. If not providing in-capsule automatic analysis and treatment, the data is transmitted to the external data collector, and optionally monitored, at least some of the time, by a medical practitioner. Based on the data, which may be similar to the data described above with reference to a separate capsule imager, the medical practitioner decides when to provide therapeutic illumination, and optionally sends a signal to a controller in the capsule to activate therapeutic light sources.

In some embodiments of the invention an automatic process of analysis, diagnosis, and provision of therapy control signals, may occur external to the capsule, using data transmitted by the capsule. In some embodiments of the invention a medical practitioner monitors the automatic process.

In some embodiments of the invention, the capsule imager records/collects images while phototherapeutic illumination is being provided, and assesses uniformity and intensity of the illumination. In some embodiments of the invention, the assessment is performed outside the capsule based on images transmitted from the capsule imager. In some embodiments of the invention, the assessment is performed in the capsule based on images transmitted from the capsule imager.

In some embodiments of the invention, the capsule imager measures intensity of the phototherapeutic illumination while the phototherapeutic illumination is ON, and calculates the dose being applied. In some embodiments the calculation of the dose takes into account the speed of the capsule in the GI tract, to calculate the dose. In some embodiments of the invention, the assessment is performed outside the capsule based on images transmitted from the capsule imager. In some embodiments of the invention, the assessment is performed in the capsule based on images transmitted from the capsule imager.

Some non-limiting potential advantages to using a combined capsule include:

When only a small area of the GI tract needs to be treated: in such a case battery power may be enough to power both imaging and phototherapy.

A patient does not have to endure two cycles of swallowing two capsules, and waiting for the two capsules to go through the GI tract. The combined capsule saves a patient's time, and may save a physician's time.

One capsule may be cheaper than two, or more. (On the other hand, a separate phototherapy capsule can be cheaper, and when several phototherapy capsules are used for treatment, using separate phototherapy capsules becomes even more financially advantageous).

In some embodiments of the invention, the combined capsule additionally includes a magnet, optionally a rare earth magnet. An external magnet, optionally an external electromagnet, is used to manipulate the combined capsule remotely. The combined capsule provides images of a diseased region in the GI tract, and/or in the stomach, and/or in the esophagus, and a medical practitioner manipulates the combined capsule to the diseased region, and provides phototherapy to the diseased region.

It is noted that when a capsule is used for treating a person who is already slated for phototherapy, there is a possibility of transmitting fewer images per second, and/or less detailed images, to an external monitor. A capsule advances rather slowly in most sections of the GI tract. When the transmitting is used for deciding when and/or where to provide phototherapy, the capsule may transmit at a lower rate, just enough to scan the GI tract, without producing multiple images of the same area. The lower transmission rate and/or image resolution may provide a power saving, which may be translated into more power for treatment.

In some embodiments of the invention, the capsule may transmit images only once every second, or even less.

In some embodiments of the invention, the capsule may transmit images only once every specific distance, such as once every centimeter, or even less, the distance being calculated by the capsule.

In some embodiments of the invention the combined capsule or capsules are optionally used to provide illumination for PDT at diseased portions of the GI tract, as detected by capsule imager.

Before explaining at least one embodiment of the invention in detail, it is to be understood that the invention is not necessarily limited in its application to the details of construction and the arrangement of the components and/or methods set forth in the following description and/or illustrated in the drawings. The invention is capable of other embodiments or of being practiced or carried out in various ways.

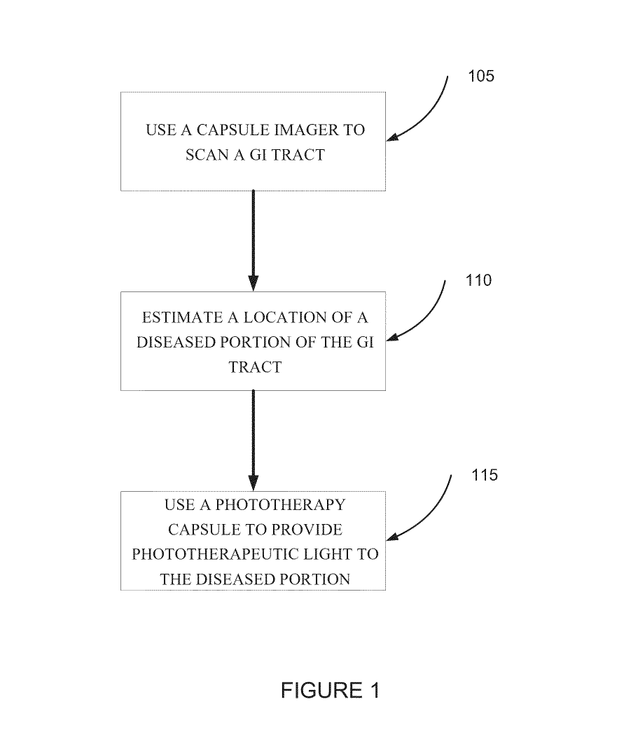

Reference is now made to FIG. 1, which is a simplified flow chart illustration of a method of providing phototherapy to a GI tract according to an embodiment of the present invention.

FIG. 1 depicts a simplified method for providing phototherapy to a GI tract including:

using a capsule imager to scan a GI tract (105);

estimating a location of a diseased portion of the GI tract (110); and

based, at least in part, on the recorded location, using a phototherapy capsule to provide phototherapeutic light to the diseased portion (115).

It is noted that the estimating a location may include an estimated error, and a treatment plan may optionally take the estimated error into account, starting treatment early and/or ending treatment late.

The above-mentioned PCT Patent Application No. PCT IL2011/000257 and a US Provisional patent application by the same inventors, having an attorney docket of 52206 and titled “CAPSULE PHOTOTHERAPY”, describe how sometimes more than one phototherapy capsule is used in order to provide treatment, typically because one phototherapy capsule might not have enough power to cover the whole diseased area.

In some cases, by finding the diseased area with a capsule imager, the diseased area is known, and it is then possible to determine how many phototherapy capsules, and of which time delay, are needed for treatment. Using this method it is possible to use one capsule imager to plan treatment by more than one phototherapy capsule. When a capsule imager is combined with a phototherapy unit in one capsule, and the mapping was unknown, in some cases it would be necessary to use more than one such combined capsule for treatment, which may be more expensive and/or more time to consuming than using a capsule imager for mapping and phototherapy capsules for treatment.

Time-Based Treatment

In some embodiments of the invention, a capsule imager is swallowed and goes through the GI tract. A medical practitioner views images acquired along the GI tract, and record at what time during the travel of the capsule imager through the GI tract disease is detected.

In some embodiments of the invention, the location of phototherapy treatment is not based on spatial coordinates, but rather on how long it took the capsule imager to reach a diseased portion of the GI tract, and/or how long it took the capsule imager to traverse the diseased portion. A potential advantage of such embodiments is that measuring time from a capsule imager is very simple—either measuring clock time from beginning of a capsule imager journey, or adding clock time to a capsule imager recording. In some embodiments of the invention measuring time for the phototherapy capsule may be done by adding a timer to the capsule, which is a simple circuit, and does not require much power. In some embodiments of the invention clock time is measured during the journey of the phototherapy capsule, and activating the capsule illumination from outside the patient's body is optionally performed based on the clock time, optionally using an activator such as an electromagnet, or ultrasound beacon signal, and so on.

The medical practitioner then programs the phototherapy capsule to provide therapeutic illumination during an interval starting from substantially the time it took the capsule imager to reach the diseased portion of the GI tract, until the capsule imager traversed to the end of the diseased portion.

In some embodiments of the invention, the interval is made to start earlier and end later than the above-mentioned interval, in order to allow for differences in actual travel speed of the capsule through the GI tract, from expected speed of travel.

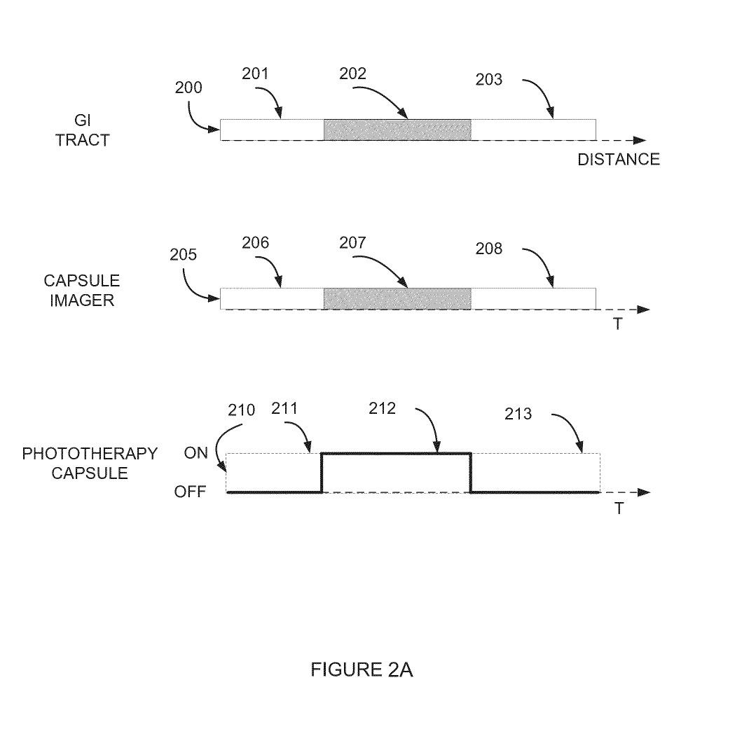

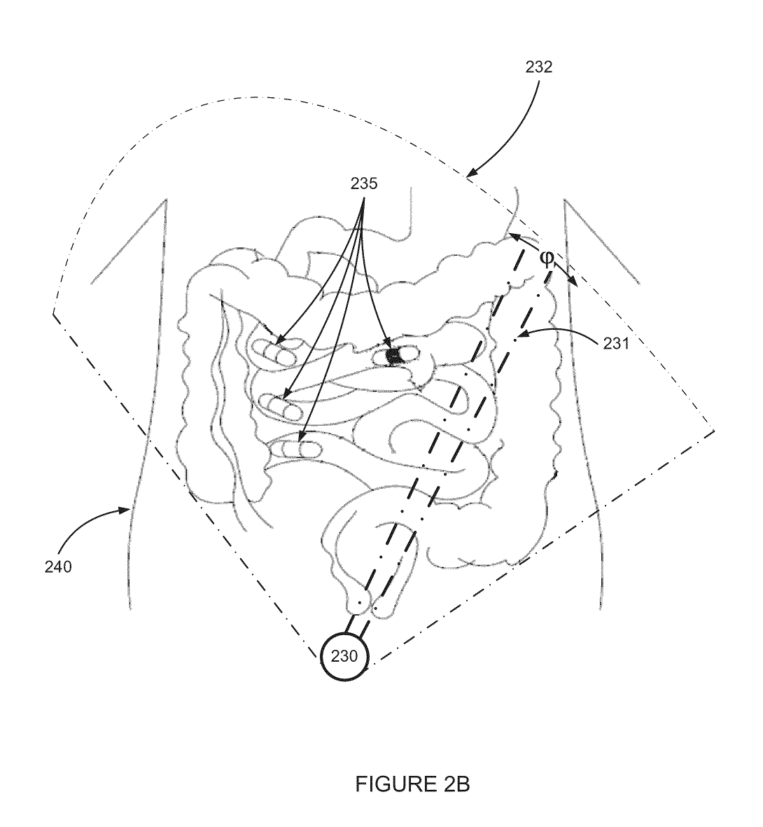

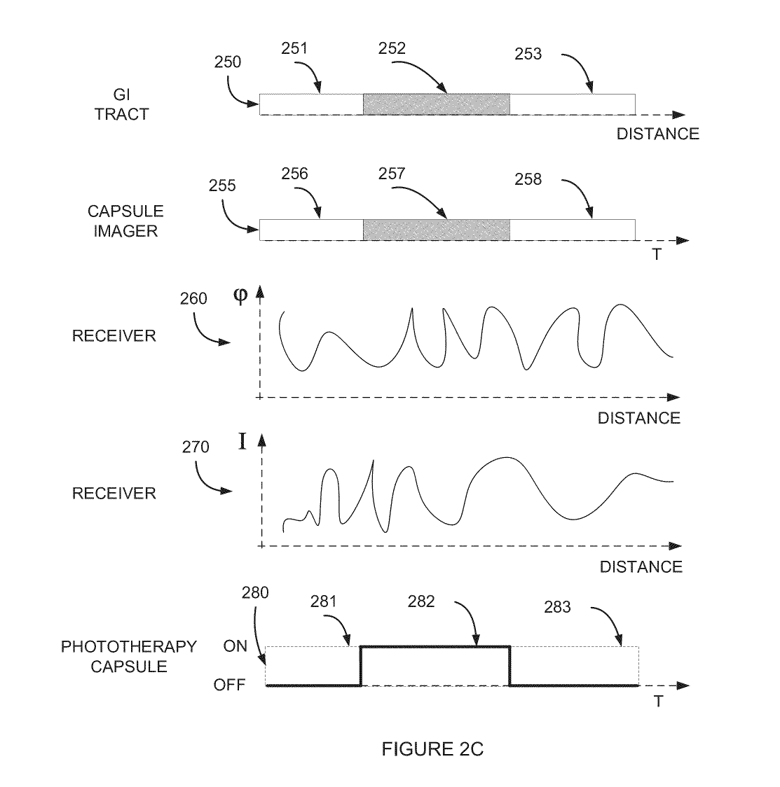

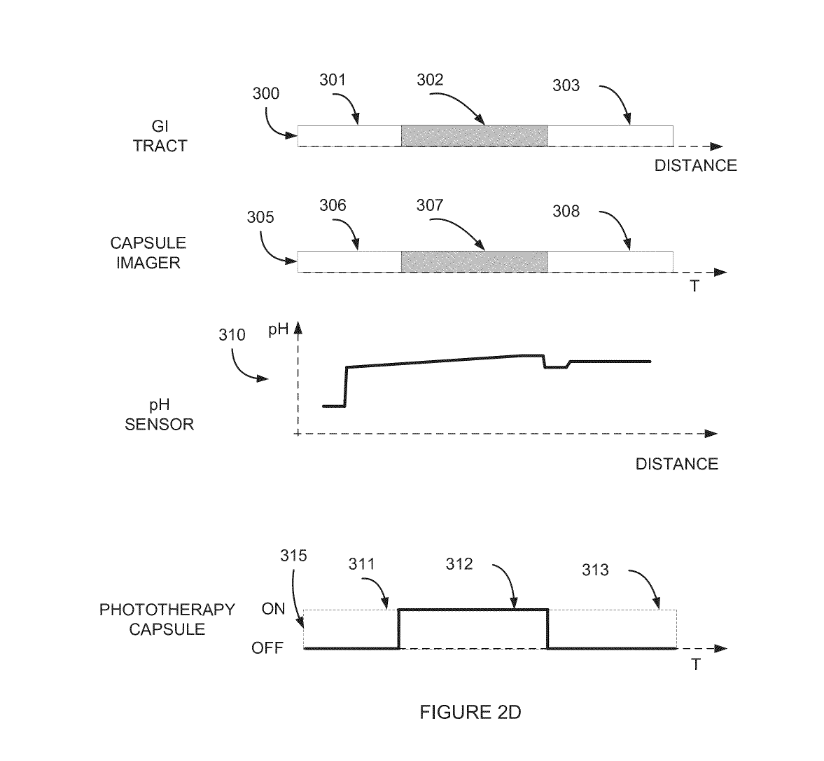

Reference is now made to FIG. 2A, which is a simplified horizontal bar graph corresponding to a length of a GI tract 200, a corresponding horizontal bar graph of capsule imager time 205 taken to journey through the GI tract 200, and a corresponding horizontal bar graph of phototherapy capsule time 210 taken to journey through the GI tract 200, according to an example embodiment of the invention.

The horizontal bar graph corresponding to a length of a GI tract 200 corresponds to spatial distance along the GI tract 200, and depicts a healthy section 201 of the GI tract 200, a diseased section 202, and a healthy section 203 again.

The corresponding horizontal bar graph of capsule imager time 205 taken to journey through the GI tract 200 corresponds to time, and depicts the pill cam displaying a healthy section for a first period of time 206, a diseased section for a second period of time 207, and a healthy section for a third period of time 208.

The corresponding horizontal bar graph of phototherapy capsule time 210 taken to journey through the GI tract 200 corresponds to time, and depicts:

a first period of time 211, corresponding to the first period of time 206 during which the capsule imager viewed a healthy section of the GI tract 200, during which the phototherapy capsule travels with its light sources OFF, not providing therapeutic illumination;

a second period of time 212, corresponding to the second period of time 207 during which the capsule imager viewed a diseased section of the GI tract 200, during which the phototherapy capsule travels with its light sources ON, providing therapeutic illumination; and

a third period of time 213, corresponding to the third period of time 208 during which the capsule imager viewed a healthy section of the GI tract 200 again, during which the phototherapy capsule travels with its light sources OFF, not providing therapeutic illumination.

In some embodiments of the invention, a medical practitioner watches the images produced by the capsule imager during its travel through the GI tract, and records each time when a diseased section of the GI tract starts and ends.

In some embodiments of the invention, the phototherapy capsule is sized, shaped, and/or weighted to be substantially similar to a capsule imager, so as to take a substantially equal time to travel through the GI tract, under the same protocol of a fluid intake regimen.

In some embodiments of the invention, it is the capsule imager which is sized, shaped, and weighted to be substantially similar to the phototherapy capsule, so as to take a substantially equal time to travel through the GI tract, under the same protocol of a fluid intake regimen.

The travel time of a photopill may be estimated using data provided by various studies such “Compartmental Transit and Dispersion Model Analysis of Small Intestine Transit Flow in Humans”, by Lawrence X. Yu, John R. Crison and Gordon L. Amidon; International Journal of Pharmaceutics, Vol. 40; 1999 and “Relationship of Gastric Emptying and Volume Changes After Solid Meal in Humans”; by Duane D. Burton, H. Jae Kim, Michael Camilleri, Debra A. Stephens, Brian P. Mullan, Michael K. O'Connor, and Nicholas J. Talley; Am J Physiol Gastrointest Liver Physiol 289, 2005. Optionally, travel times for a given patient are estimated from measurements of travel times of objects through the GI tract of the patient.

In some embodiments of the invention, when the times are known, the medical practitioner programs the phototherapy capsule, setting times when the capsule lights will turn ON and OFF.

In some embodiments of the invention, the medical practitioner programs the phototherapy capsule wirelessly, possibly even after the capsule has been swallowed.

In some embodiments of the invention, when the times are known, the medical practitioner selects a phototherapy capsule which is pre-programmed with times when the capsule lights will turn ON and OFF.

In some embodiments of the invention a patient may swallow the phototherapy capsule in a non-clinic setting, even at home.

Location Based Treatment

In some embodiments of the invention, a capsule imager is swallowed and goes through the GI tract. A medical practitioner views images acquired along the GI tract, and records at what location during the travel of the capsule imager through the GI tract disease is detected.

In some embodiments of the invention, a capsule imager includes a same sensor type as a phototherapy capsule, e.g. a pH sensor, and/or an accelerometer. A location as determined by using the sensor(s) of the capsule imager can be reached by using the same sensor to determine location of the phototherapy capsule.

In some embodiments of the invention, a capsule imager provides location of a diseased portion of the GI tract arrived at by using any one or more of several types of sensor(s) (for example by measuring time following a pH change), and location of the phototherapy capsule is arrived at by using any one or more of other types of sensor(s), (for example by calculating location based on an accelerometer) at least some of which may be different from the sensor(s) of the capsule imager. Some non-limiting examples of the above scenario include: measuring time of capsule imager travel and measuring location of the one or more types of sensor(s); measuring location of the capsule imager and measuring time of phototherapy travel; measuring location of capsule imager using an accelerometer and measuring location of the phototherapy capsule using medical imaging.

It is noted that generally speaking, the pH sensors, accelerometer and temperature changes are rough sensors, which may have a relatively high measurement error, while the optical and imager sensors are finer, more accurate sensors. In some embodiments of the invention, a rough sensor, such as a pH sensor, is used to detect travel into a certain area of the GI tract, for example—passage from the stomach to the small intestine, and then a finer sensor, such as an imager, is used to measure advance and/or speed within the area.

Different embodiments of location determination for a capsule are now described, the embodiments holding true for both a capsule imager and a phototherapy capsule.

In some embodiments of the present invention, a capsule includes mechanisms for determining its direction, and/or acceleration, and/or speed of movement, and/or location as it travels through the GI tract. The above data is optionally used to calculate location.

In some embodiments, the speed of movement is provided by an optical motion sensing system, generally comprising an illumination source (optionally shared with a phototherapy illumination source) and one or more photo-detectors disposed within the capsule, such that the photo-detectors detect light signals emitted by the illumination source and reflected back towards the capsule by a structure, such as the intestinal wall. A non-limiting example of how to compute speed of movement by an optical sensing system includes a comparison of different image frames separated by time, optionally using normalized cross-correlation, to measure speed in pixels per time separation. By knowing optical specifications of the camera and an image rate (e.g. frames-per-second), speed of movement may be translated to distance per time unit. Another non-limiting example of how to compute speed of movement by an optical sensing system includes using a mechanism similar to a computer optical mouse.

In some embodiments of the invention, changes in speed are used to determine where the capsule is located in the GI tract. In some embodiments of the invention, location is determined by double-integrating acceleration determined responsive to measurements by an accelerometer, over time, to determine distance traveled through the GI tract.

In some embodiments of the invention, a substantial change in direction of the capsule, optionally indicated by acceleration measurements provided by the accelerometer, is used to determine location.

In some embodiments of the invention, a substantial change in speed, optionally indicated by acceleration measurements provided by the accelerometer, is used to determine location. For example, material propagating through the GI tract moves more slowly in the Cecum than in the small intestine. As a result, a substantial decrease in speed of travel of the capsule indicated by accelerometer signals, indicating substantial deceleration, is optionally used to determine when the photopill reaches the Cecum.

In some embodiments of the invention, location of a capsule may be determined using any of various medical imaging modalities, such as X-ray computerized tomography (CT) and ultrasound imaging.

In some embodiments of the invention, an acoustically reflective calibration capsule is swallowed by the patient and progress of the calibration capsule through the patient's GI tract is measured using ultrasound sensors and/or wireless transmission of an internal acceleration and/or speed and/or location measurement to an external recorder.

Optionally “travel distance” is determined by double integrating acceleration measurements provided by the accelerometer.

Reference is now additionally made to FIG. 2B, which is a simplified illustration of a directional beacon 230 providing signals to a patient's body 240, according to an example embodiment of the present invention.

FIG. 2B schematically shows a capsule 235 in several locations of a patient's GI tract 242. The directional beacon 230 is optionally mounted at a known location external to the patient's body. By way of example, the directional beacon 230 is shown close to the patient's body in a region of the lower abdomen. The beacon may be placed on an abdomen, as in an ultrasound probe.

The directional beacon 230 transmits a beam 231, sweeping an area 232. The beam may optionally be of acoustic energy, such as ultrasound. The beam is modulated such that its frequency “f” changes respectively with an angular direction “φ” along which the beam is transmitted and its intensity “I” with a radial distance “r” away from the beacon.

A location of a region in the patient's body may be determined by determining what frequency and intensity of the beam 231 are received at the location.

In some embodiments of the invention a body is “calibrated”. A “calibration capsule” is swallowed by a calibration patient, and results of receiving the beam 231 are mapped.

In some embodiments of the invention a lookup table is used to map a frequency f(φ) and intensity I(r) of the beam 231 to locations in a patient's body.

Reference is now made to FIG. 2C, which is a simplified horizontal bar graph corresponding to a length of a GI tract 250, a corresponding horizontal bar graph of capsule imager time 305 taken to journey through the GI tract 255, a corresponding graph 260 of frequency “φ” of a directional beacon as measured by a capsule journeying through the GI tract 250, a corresponding graph 270 of intensity “I” of the directional beacon as measured by a capsule journeying through the GI tract 250, and a corresponding horizontal bar graph of phototherapy capsule time 280 taken to journey through the GI tract 250.

The horizontal bar graph corresponding to a length of a GI tract 250 corresponds to spatial distance along the GI tract 250, and depicts a healthy section 251 of the GI tract 300, a diseased section 252, and a healthy section 253 again.

The corresponding horizontal bar graph of capsule imager time 255 taken to journey through the GI tract 250 corresponds to time, and depicts the capsule imager displaying a healthy section for a first period of time 256, a diseased section for a second period of time 257, and a healthy section for a third period of time 258.

The corresponding graph 260 of frequency “φ” of the directional beacon as measured by a capsule journeying through the GI tract 250, depicts “φ” as measured by a receiver in the capsule imager in its travel through the GI tract 250.

A horizontal axis of the graph 260 depicts time of travel along the GI tract 250, and corresponds to a horizontal axis of the horizontal bar graph corresponding to a length of a GI tract 250, and a vertical axis of the graph 260 depicts a qualitative “φ” value as measured in the GI tract.

The graph 260 is a qualitative graph, drawn to emphasize that different locations in the GI tract provide different “φ” values.

The corresponding graph 270 of intensity “I” of the directional beacon as measured by a capsule journeying through the GI tract 250, depicts “I” as measured by a receiver in the capsule imager in its travel through the GI tract 250.

A horizontal axis of the graph 270 depicts distance along the GI tract 250, and corresponds to a horizontal axis of the horizontal bar graph corresponding to a length of a GI tract 250, and a vertical axis of the graph 270 depicts a qualitative “I” value as measured in the GI tract.

The graph 270 is a qualitative graph, drawn to emphasize that different locations in the GI tract provide different “I” values.

While values of frequency “φ” may repeat along the GI tract, and values of intensity “I” may repeat along the GI tract, a combination of a specific value of frequency “φ” and a specific value of “I” is less frequently repeated. It is noted that travel within the GI tract occurs in a certain order, constrained by the shape of the GI tract.

In some embodiments of the invention, a medical practitioner watches the images produced by the capsule imager during its travel through the GI tract, and records each time when a diseased section of the GI tract starts and ends. The medical practitioner, and/or an automatic program, also records a specific value of frequency “φ” and a specific value of “I” as measured by the capsule imager, when the diseased section of the GI tract starts and ends.

The medical practitioner then programs a phototherapy capsule, or selects a pre-programmed capsule, to start measuring time and/or location starting from a specific combination of frequency “φ” and a specific value of “I” made by the capsule imager, and optionally a specific time.

The corresponding horizontal bar graph of phototherapy capsule time 280 taken to journey through the GI tract corresponds to time, and depicts:

a first period of time 281, corresponding to the first period of time 251 during which the capsule imager viewed a healthy section of the GI tract 250, during which the phototherapy capsule travels with its light sources OFF, not providing therapeutic illumination;

a second period of time 282, corresponding to the second period of time 252 during which the capsule imager viewed a diseased section of the GI tract 250, during which the phototherapy capsule travels with its light sources ON, providing therapeutic illumination; and

a third period of time 283, corresponding to the third period of time 253 during which the capsule imager viewed a healthy section of the GI tract 250 again, during which the phototherapy capsule travels with its light sources OFF, not providing therapeutic illumination.

Treatment Based on Additional Sensors

In some embodiments of the invention, a capsule imager is swallowed and goes along the GI tract. A medical practitioner views images acquired along the GI tract, and records at what location during the travel of the capsule imager through the GI tract disease is detected. At the same time, the capsule imager provides data about additional GI tract environmental factors.

In some embodiments of the invention, the capsule imager provides pH measurements from the GI tract.

Since pH values change significantly along the GI tract, the location of a capsule can be verified by the capsule measuring pH. The following table provides minimum and maximum pH values typically found in the various regions of the GI tract in human subjects:

| Location | Min pH | Max pH |
|---|---|---|
| Stomach | 1.0 | 2.5 |
| Proximal small Intestine | 6.1 | 7.1 |
| Terminal ileum | 7.1 | 7.9 |
| Caecum | 6.0 | 6.8 |
| Left Colon | 6.3 | 7.7 |

It may thus be appreciated that an ambient pH value can be used to estimate a passage of a capsule from one section of the GI tract into another.

Measurement of pH can be performed using a pH sensor incorporated within the capsule. While any suitable sensor can be used, in one embodiment, the pH sensor may be an ISFET (ion sensitive field effect transistor) sensor, such as the sensor used in a telemetry capsule described in US 2004/0106849.

In some embodiments of the invention, detecting a time at which a capsule imager passes from one section of the GI tract into another, based on an indication such as change of pH, or change of speed, optionally serves for recording a time and/or location which is closer to a start of a diseased section of the GI tract.

From the time or location of passage onwards, other mechanisms, such as time and/or the accelerometer and/or optical motion sensor described hereinabove, are optionally used in order to measure passage to a diseased section within the GI tract.

The closer time and/or location serve to start measurement of time and/or location by the phototherapy capsule. Starting measurement closer to a diseased section can reduce errors in the starting and ending of phototherapeutic illumination.

Reference is now made to FIG. 2D, which is a simplified horizontal bar graph corresponding to a length of a GI tract 300, a corresponding horizontal bar graph of capsule imager time 305 taken to journey through the GI tract 300, a corresponding graph of pH 310 as measured by a capsule journeying through the GI tract 300, and a corresponding horizontal bar graph of phototherapy capsule time 315 taken to journey through the GI tract 300, according to an example embodiment of the invention.

The horizontal bar graph corresponding to a length of a GI tract 300 corresponds to spatial distance along the GI tract 300, and depicts a healthy section 301 of the GI tract 300, a diseased section 302, and a healthy section 303 again.

The corresponding horizontal bar graph of capsule imager time 305 taken to journey through the GI tract 200 corresponds to time, and depicts the pill cam displaying a healthy section for a first period of time 306, a diseased section for a second period of time 307, and a healthy section for a third period of time 308.

The corresponding graph of pH 310 as measured by a capsule journeying through the GI tract 300, depicts pH as measured by the pill cam in its travel through the GI tract.

A horizontal axis of the graph of pH 310 depicts distance along the GI tract, and corresponds to the horizontal axis of the horizontal bar graph corresponding to a length of a GI tract 300, and a vertical axis of the graph of pH 310 depicts a qualitative pH value as measured in the GI tract.

The graph of pH 310 is a qualitative graph, drawn to emphasize that some locations in the GI tract provide pH transition which may assist in locating a capsule during its travel through the GI tract.

In some embodiments of the invention, a medical practitioner watches the images produced by the capsule imager during its travel through the GI tract, and records each time when a diseased section of the GI tract starts and ends. The medical practitioner, and/or an automatic program, optionally also records pH as measured by the capsule imager when the diseased section of the GI tract starts and ends.

The medical practitioner then programs a phototherapy capsule, or selects a pre-programmed capsule, to start measuring time and/or location starting from a specific pH reading made by the capsule imager capsule, and/or a specific inflection point in a pH reading made by the capsule imager.

The corresponding horizontal bar graph of phototherapy capsule time 315 taken to journey through the GI tract 300 corresponds to time, and depicts:

a first period of time 311, corresponding to the first period of time 306 during which the capsule imager viewed a healthy section of the GI tract 300, during which the phototherapy capsule travels with its light sources OFF, not providing therapeutic illumination;

a second period of time 312, corresponding to the second period of time 307 during which the capsule imager viewed a diseased section of the GI tract 300, during which the phototherapy capsule travels with its light sources ON, providing therapeutic illumination; and

a third period of time 313, corresponding to the third period of time 308 during which the capsule imager viewed a healthy section of the GI tract 300 again, during which the phototherapy capsule travels with its light sources OFF, not providing therapeutic illumination.

Total Treatment Energy

The therapeutic light emitted by the capsule is optionally energized by an internal battery located within the capsule. A theoretical maximal energy, to be supplied by the battery, may be calculated as follows:

Bat_total_power=I·V Equation 1

where I is a continuous drain current from the battery, and V is the average battery voltage (battery voltage drops as the battery is exhausted). Battery capacity is measured by mAH (milli-Ampere Hour), so the battery capacity determines a duration during which a given current can be drained from the battery.

Battery theoretical energy can be defined by total Joule according to:

E=Bat_total_power·Discharge_time hd Equation 2

A maximal theoretical treated area is optionally calculated by using the required energy density (in Joule/cm2) for treatment, compared with a maximal theoretical energy provided by the battery:

Treat_area=E/Dose Equation 3

A therapeutic digestible capsule is often limited by its capacity. The capsule cannot use large sized batteries.

By way of a typical example: an 80 mAh battery is used with a discharge voltage curve averaging at 3.6V. Using the formulas above, the maximal theoretical treated area:

Treat_area=V·I·T/Dose Equation 3:

| | Dose | | | |
|---|---|---|---|---|
| | 1 J/cm² | 0.5 J/cm² | 0.25 J/cm² | 0.1 J/cm² |
| Treat Area | 1,036 cm² | 2,073 cm² | 4,147 cm² | 10,360 cm² |

Due to losses from elements in electronic circuits in the capsule, and from therapeutic LEDs, the “real life” treated area may be lower. Some contributors to energy “loss” are:

DC to DC conversion and LED drivers, typical loss of about 15%;

LED efficiency, typical loss of about 70%; and

control electronics, typical loss of about 5%.

It is noted that LED efficacy is dependent on a dominant wavelength of the LED. Currently available Near Infra Red (NIR) or Red LEDs can show efficiency as stated above, 20%-30%. LEDs at other wavelengths can demonstrate a lower efficiency, such as a Blue LED which can demonstrate typical efficiency of 15%-20%.

In reality, a theoretical treatment area corresponding to 25% of the battery maximal energy is expected.

A simulation of the actual capsule performance has been conducted, yielding the following results for “real life” capsule treatment:

For a NIR LED treatment source—

| | Dose | | | |
|---|---|---|---|---|
| | 1 J/cm² | 0.5 J/cm² | 0.25 J/cm² | 0.1 J/cm² |
| 3 H peristaltic | 218 cm² | 431 cm² | 885 cm² | 2,212 cm² |
| 4 H peristaltic | 216 cm² | 433 cm² | 873 cm² | 2,164 cm² |

The simulation presented above is based on a capsule with a diameter of 11 mm, and an average small intestine of 15 mm diameter. Smaller intestine radius results in increase treatment area covered by the LEDs.

Area of Treatment

Typically, Crohn's Disease (CD) is a complete GI tract disease (“mouth to anus”). In most patients, Crohn's disease involves the small intestine, and in some patients, the large intestine is involved as well.

As most cases of CD involve the small intestine in general, and the terminal ileum in particular, a purpose of a capsule, as implemented in some embodiments of the invention, is to illuminate the entire small intestine if possible. If limited, for example by a limitation of treatment area, to partial coverage of the small intestine, then the terminal ileum is a priority area for treatment.

A typical entire small intestine is about 7 meters (700 centimeters) in length, with an average internal diameter of about 1.5 cm-2 cm. The terminal ileum length is about 2 m-4 m.

Using those dimensions, a treatment area can be calculated based on a simplified model of a tube:

Area=2·π·r·h Equation 4

Based on the above, an entire small intestine average treated area is approximately 6,594 cm2, and the terminal ileum treated area is approximately 2,826 to cm2.

In some embodiments of the invention, a capsule used in the small intestine, which has an average internal diameter of the intestine walls of 15 mm, is only slightly smaller than the intestine diameter, or approximately 9 mm-11 mm. In most cases, during the peristaltic movement, the intestine walls are “collapsed” on the capsule, or very close to the capsule, which forces the capsule's orientation to be heading down the intestine with substantially little to no side movements.

Use of NIR wavelength allows for deep tissue penetration, up to 4 cm, which overcomes a need to have treated tissue in direct sight of the LED sources. Deep tissue penetration is associated with NIR and IR, and is reduced when wavelength shortens from Yellow to Blue and UV. The deep tissue penetration aids a capsule phototherapy to prevent folds in the intestine from interfering with phototherapeutic delivery to the intestine tissue.

An overlap of areas of treatment may cause an over dose of a certain overlap area and, as explained before, will show less effectiveness in this area. The activation mechanism of the light sources optionally acts to keep those overlap areas to a minimum.

Adjustment of Illumination

In some embodiments of the invention, the phototherapy capsule optionally adjusts a phototherapeutic dose by switching the light sources off, then back on, providing an accurate therapeutic dose as needed.

In some embodiments of the invention, the phototherapy capsule optionally decreases or increases a phototherapeutic dose by adjusting the intensity of the light sources, providing an accurate therapeutic dose as needed.

In some embodiments of the invention, the above-mentioned adjustments of illumination take into account the speed of the phototherapy capsule, optionally as measured by the capsule as described above.

In some embodiments of the invention, the above-mentioned adjustments of illumination take into account the speed of the phototherapy capsule, optionally as measured outside the capsule, by an imaging system, optionally communicated to the phototherapy capsule.

Packaging a Capsule Imager in a Phototherapy Capsule

As mentioned above, in some cases it is useful to package capsule imager functions together with capsule phototherapy functions in one capsule. For example, such cases may occur when only a small area of the GI tract needs to be treated. In such a case battery power may be enough to power both imaging and phototherapy. An advantage of the combined package is that a patient does not have to endure two cycles of swallowing two capsules, and waiting for the two capsules to go through the GI tract. The combined capsule saves a patient's time, and may save a physician's time.

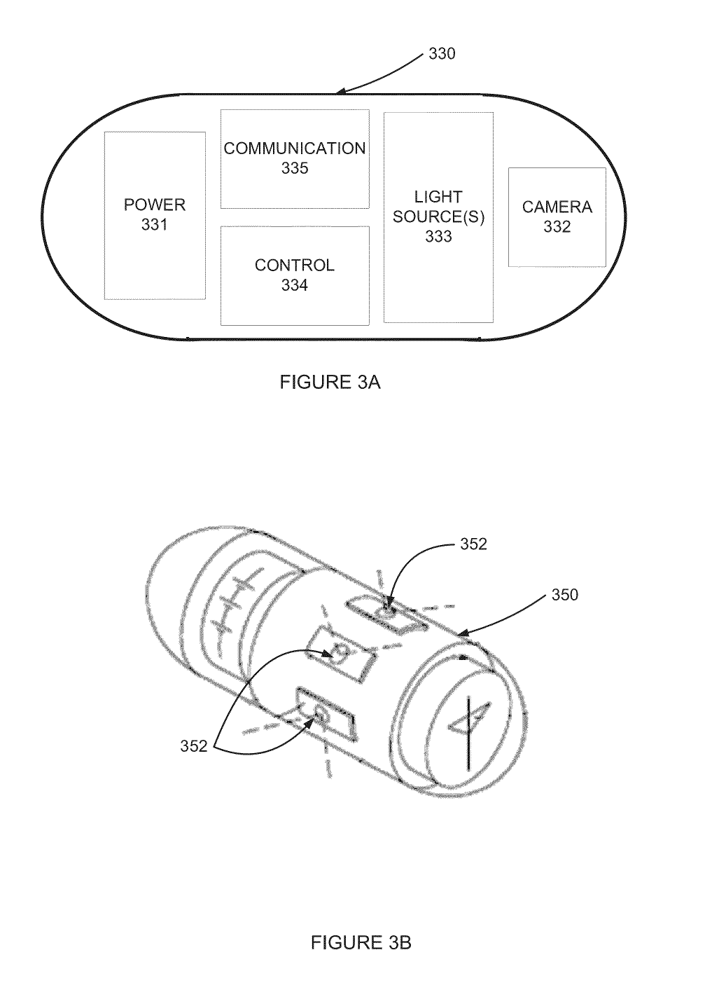

Reference is now made to FIG. 3A, which is a simplified block diagram illustration of a swallowable combination capsule 330 for providing capsule endoscopy and phototherapy to a GI tract, constructed according to an example embodiment of the invention.

The combination capsule 330 includes a power supply 331, a camera unit 332, one or more light sources 333, a control unit 334 for activating one or more of the one or more light sources 333, and a communication unit 335 for transmitting images from the camera unit to an out-of-body receiver and for receiving control commands from an out-of-body transmitter.

The power supply 331, the camera unit 332, the one or more light sources 333, the control unit 334, and the communication unit 335 are functionally connected as may be expected by a person skilled in the art.

Reference is now made to FIG. 3B, which is another simplified block diagram illustration of a swallowable combination capsule 350 for providing capsule endoscopy and phototherapy to a GI tract, constructed according to an example embodiment of the invention.

The capsule 350 contains the elements described above with reference to FIG. 3A. The one or more light sources 352 of the example embodiment of FIG. 3B are each configured to provide light to a substantially limited angle around a longitudinal axis of the capsule, as may be seen in FIG. 3B.

In the embodiment of FIG. 3B the control unit (not shown) is configured to turn each one of the light sources off and/or on separately and simultaneously, thereby producing phototherapeutic illumination only to some of the circumference of the capsule 350. Producing illumination for only part of the circumference can optionally save power, and target a diseased area based on results from the camera unit.

In some embodiments of the invention a medical practitioner watches images communicated from the camera unit, and commands, via the communication unit, to the control unit, to determine which sector of the circumference of the capsule 350 receives phototherapeutic illumination.

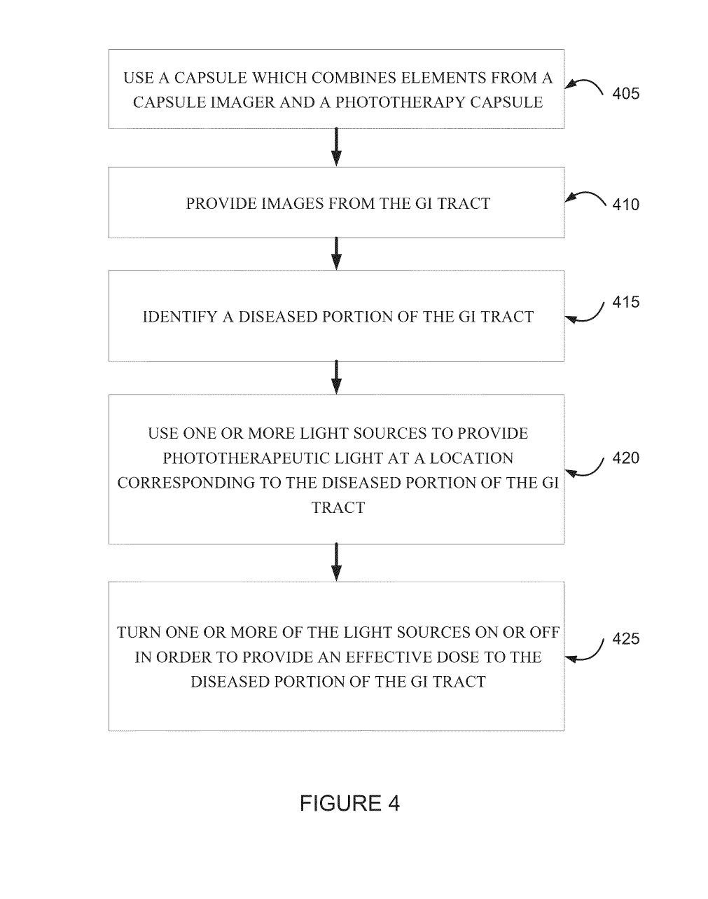

Reference is now made to FIG. 4, which is a simplified flow chart illustration of a method of providing phototherapy to a GI tract according to an example embodiment of the present invention.

FIG. 4 depicts a simplified method for providing phototherapy to a GI tract including:

using a capsule which combines elements from a capsule imager and a phototherapy capsule (405);

providing images from the GI tract (410);

identifying a diseased portion of the GI tract (415);

using one or more light sources to provide phototherapeutic light at a location corresponding to the diseased portion of the GI tract, based, at least in part, on the images (420); and

in which the one or more light sources are turned on or off in order to provide an effective dose to the diseased portion of the GI tract (425).

In some embodiments of the invention, as described above with reference to FIG. 3B, the one or more light sources comprise a plurality of light sources, each providing light to a substantially limited angle around a longitudinal axis of the capsule, and providing the therapeutic light involves causing at least one of the light sources to be off, while causing at least one of the light sources to be off, based, at least in part, on identifying whether the limited angle which the light source illuminates includes a diseased portion of the GI tract.

In some embodiments of the invention, a medical practitioner sees which directions around the phototherapy capsule are diseased, and activates only light sources illuminating the diseased directions.

In some embodiments of the invention, as described above in a section named “Location based treatment” the one or more light sources are turned on or off based, at least in part, on speed of movement of the capsule in the GI tract.

In some embodiments of the invention, as described above in a section named “Adjustment of illumination” the intensity of a light source is adjusted, based, at least in part, on speed of movement of the capsule in the GI tract.

Automatic Detection and Optional Automatic Treatment

In some embodiments of the invention, the capsule analyzes inputs from a video camera in the capsule, determining when the phototherapy capsule is in a diseased portion of the GI tract, and automatically activates light sources illuminating the diseased tissue in the diseased portion. The above embodiments are suitable for treating diseases which present a discoloration of the GI tract, and/or which are suitable for automatic detection by image analysis and/or by multi-spectral analysis, optionally using multi-spectral input sensors.

In some embodiments of the invention, the capsule analyzes inputs from a video camera in the capsule, determining which directions around the phototherapy capsule are diseased, and automatically activates only light sources illuminating the diseased directions.

In some embodiments of the invention, the capsule analyzes inputs from a video camera in the capsule, detecting disease when the color of the GI wall is red.

In some embodiments of the invention, the capsule analyzes inputs from a video camera in the capsule, detecting disease when the images show bleeding.

In some embodiments of the invention, the capsule analyzes inputs from a video camera in the capsule, detecting disease when the images show inflammation.

By way of a non-limiting example, automatic lesion detection is performed on images captured by the capsule imager. In some embodiments of the invention, when using a combined capsule, treatment is performed automatically, turning phototherapy ON when lesions are detected. In some embodiments of the invention, when using a separate capsule imager, a treatment program is automatically generated for a phototherapy capsule.

In some embodiments of the invention, the capsule analyzes tissue color inputs from a video camera in the capsule, and automatically activates light sources for treating specific tissue color.

In some embodiments of the invention, the capsule detects local bleeding seen in images from a video camera in the capsule, and automatically activates light sources for treating specific tissue color.

Treatment Scenarios

Some treatment scenarios envisaged for embodiments of the present invention are now additionally described.

In a scenario where a separate capsule imager and a separate phototherapy capsule are used, a clinic may administer a capsule imager, collect images and possible data from the capsule imager, and a medical practitioner may program the phototherapy capsule to provide illumination, based on a timer, a location, and additional inputs as described above.

In a scenario where a separate capsule imager and a separate phototherapy capsule are used, a clinic may administer a capsule imager, collect images and possible data from the capsule imager, and a medical practitioner may turn phototherapeutic illumination provided by a phototherapy capsule on or off based on location of the phototherapy capsule, as determined by time and/or by an medical imaging system, such as, by way of a non-limiting example ultrasound.

In a scenario where a combined capsule which features capsule imager and phototherapy is used, a clinic may administer the combined capsule, and a medical practitioner may instruct the combined capsule to provide phototherapeutic illumination, based on images from the combined capsule.

In a scenario where a combined capsule which features capsule imager and phototherapy is used, the medical practitioner may adjust intensity of the phototherapeutic light, and/or turn the light sources on and off, based on viewing an image stream from the capsule and/or based on data such as speed of movement of the capsule in the GI tract.

In a scenario where a combined capsule which features capsule imager and phototherapy is used, the medical practitioner may turn on phototherapeutic illumination only in a specific angle relative to longitudinal axis of the capsule, based on viewing an image stream from the capsule.

In a scenario where a combined capsule which features capsule imager and phototherapy is used, a clinic may administer several combined capsules to several patients, and a medical practitioner may control treatment of several patients simultaneously, by viewing several image streams, and optionally instruct several combined capsules to provide phototherapeutic illumination. The viewing and the instructing may optionally be centralized at a console where the medical practitioner views and controls treatment for several patients.

It is expected that during the life of a patent maturing from this application many relevant capsule imagers and phototherapy capsules will be developed and the scope of the terms capsule imager and phototherapy capsule is intended to include all such new technologies a priori.

The terms “comprising”, “including”, “having” and their conjugates mean “including but not limited to”.

The term “consisting of” is intended to mean “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a unit” or “at least one unit” may include a plurality of units, including combinations thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. cm What is claimed is:

1. A method for providing phototherapy to a GI tract comprising:
using a capsule imager to scan at least a portion of a GI tract;
estimating a location of a diseased portion of the GI tract; and
based, at least in part, on the estimated location, using a phototherapy capsule to provide phototherapeutic light to the diseased portion.

2. The method according to claim 1, in which a medical practitioner programs the phototherapy capsule to provide phototherapeutic light to the diseased portion based, at least in part, on the estimating a location providing a time required for the phototherapy capsule to reach a diseased portion of the GI tract.

3. The method according to claim 1, in which the estimating a location is performed automatically, and further comprising automatically programming the phototherapy capsule to provide phototherapeutic light to the diseased portion based, at least in part, on the estimating a location.

4. The method according to claim 3, in which the estimating a location comprises automatic lesion detection in images captured by the capsule imager.

5. The method according to claim 3, in which the estimating a location comprises analyzing tissue color in images captured by the capsule imager.

6. The method according to claim 3, in which the estimating a location comprises detecting local bleeding in images captured by the capsule imager.

7. The method according to claim 1 in which the using a capsule imager and the estimating a location of a diseased portion of the GI tract are performed in a medical clinic, and the using a phototherapy capsule to provide phototherapeutic light to the diseased portion is performed by a patient in a non-clinic setting.

8. The method according to claim 1, in which the phototherapy capsule determines when to turn on and off one or more phototherapeutic light sources based, at least in part, on the estimating a location providing a time required for the phototherapy capsule to reach a diseased portion of the GI tract.

9. The method according to claim 8, wherein:
the capsule imager further records data about the environment in the GI tract;
the phototherapy capsule measures data about the environment in the GI tract;
the phototherapy capsule determines when to turn on and off one or more phototherapeutic light sources based, at least in part, on the data about the environment which the capsule imager recorded and the phototherapy capsule measures.

10. The method according to claim 9 in which measuring data about the environment in the GI tract comprises at least one selected from a group consisting of:
pH measurement;
measuring ultrasound from an ultrasound beacon;
measuring sound from a sonic beacon.

11. The method according to claim 1 wherein the data recorded includes location data, and in which the phototherapy capsule determines when to turn on and off one or more phototherapeutic light sources based, at least in part, on the phototherapy capsule reaching a location of a diseased portion of the GI tract.

12. The method according to claim 1, in which the phototherapy capsule determines when to turn on and off one or more phototherapeutic light sources in order to provide a required treatment energy to the diseased portion of the GI tract.

13. The method according to claim 12, in which the required treatment energy is computed, at least in part, based on speed of movement of the phototherapy capsule in the GI tract.

14. The method according to claim 12, in which the phototherapy capsule adjusts an intensity of a light source based, at least in part, on the phototherapy capsule measuring a speed of movement of the phototherapy capsule in the GI tract.

15. The method according to claim 12, in which the phototherapy capsule uses the capsule imager to measure an intensity of the phototherapeutic light provided to the diseased portion.

16. The method according to claim 1, in which the providing phototherapeutic light to the diseased portion comprises instead providing light for PhotoDynamic Therapy (PDT).

17. A swallowable capsule for providing phototherapy to a GI tract comprising:
a power supply;
a camera unit;
one or more light sources emitting in a range including visible and Near Infra Red (NIR); and
a control unit for calculating an effective dose to the GI tract by controlling the one or more light sources;
a communication unit for transmitting images from the camera unit to an out-of-body receiver and for receiving control commands from an out-of-body transmitter.

18. The capsule according to claim 17 and further comprising a communication unit for transmitting images from the camera unit to an out-of-body receiver and for receiving control commands from an out-of-body transmitter.

19. A method for providing phototherapy to a GI tract comprising:
using a capsule which comprises a capsule imager and a phototherapy capsule, to provide images from the GI tract;
identifying a diseased portion of the GI tract;
using one or more light sources to provide phototherapeutic light at a location corresponding to the diseased portion of the GI tract, based, at least in part, on the images,
in which the one or more light sources are controlled in order to provide an effective dose to the diseased portion of the GI tract.

20. The method according to claim 19, in which the identifying is performed automatically, and further comprising automatically programming the phototherapy capsule to provide phototherapeutic light to the diseased portion based, at least in part, on the identifying.
